(19) United States
(12) Patent Application Publication
(10) Pub. No.: US 2024/0279274 A1
(43) Pub. Date: Aug. 22, 2024

(54) SYSTEM AND METHOD FOR ISOLATION AND PURIFICATION OF EXOSOMES FROM MILK

(71) Applicant: Jennifer Huang, Buffalo Grove, IL (US)

(72) Inventor: Jennifer Huang, Buffalo Grove, IL (US)

(21) Appl. No.: 18/583,214

(22) Filed: Feb. 21, 2024

Related U.S. Application Data
(60) Provisional application No. 63/447,046, filed on Feb. 21, 2023.

(51) Int. Cl. C07K 1/36; C07K 1/30; C07K 1/34

(52) U.S. Cl. CPC C07K 1/36; C07K 1/30; C07K 1/34

(57) ABSTRACT

The present invention discloses integrated systems and methods for fractionating milk components and isolating milk-derived exosomes. Caseins are separated by pH-adjusted precipitation and removal using centrifugation or filtration. The casein-depleted clear supernatant then undergoes tangential flow filtration (TFF) with specific molecular weight cut off membranes that concentrate and purify exosomes in the retentate while allowing passage of whey proteins. Residual lipoproteins are optionally subsequently removed using tailored hydrophobic interaction chromatography leveraging differential hydrophobicity between the single layer lipoprotein surface and the bilayer exosome envelope. The final exosome product stream may be concentrated and buffer exchanged using TFF into an optimized storage formulation.

### FIELD OF THE DISCLOSURE

The present disclosure relates to a method and system for isolating and purifying milk-derived exosomes. Specifically, the invention combines pH-driven casein precipitation, tangential flow filtration for concentrating exosomes and removing proteins, and hydrophobic interaction chromatography for separating lipoproteins, to efficiently recover highly purified milk exosomes.

### BACKGROUND OF THE INVENTION

Extracellular vesicles (EVs), including exosomes, are homogenous vesicles surrounded by a lipid bilayer and are naturally produced by almost all living cells. Exosomes represent a specific subtype of secreted EVs and contain a specific set of proteins, lipids, and nucleic acids. With sizes ranging from 30-150 nm in diameter, exosomes play a vital role in intercellular communication. In recent years, exosomes have showed remarkable potential as biomarkers, new therapeutic modalities, and drug delivery agents. Like exosomes derived from other sources, milk-derived exosomes (MDEs) have a lipid bilayer membrane and an aqueous core, possessing the ability to deliver both hydrophilic and hydrophobic biomolecules across biological barriers.

Due to increasing interest in exosomes as therapeutic agents and drug delivery vesicles, the production and isolation of exosomes through cell culture of stem cells or HEK sources have been areas of growing research. However, production processes are often expensive.

Milk-derived exosomes (MDEs), particularly bovine milk-derived exosomes, have been proposed as a viable alternative due to their safety, scalability, bioavailability and biocompatibility. Since bovine milk is already commercially available, existing milk treatment infrastructure allows MDEs to be isolated at a large scale. Additionally, because many people have consumed milk and other dairy products containing MDEs throughout the decades, it is likely that humans have developed an immune tolerance to many of the proteins found in bovine milk (Feng et al., 2021).

Isolating exosomes from bovine milk is extremely challenging. Bovine milk, as with other milk, is made up of a variety of components, including water, carbohydrates, fats, proteins, vitamins, and minerals. The main carbohydrate found in bovine milk is lactose, a disaccharide made up of glucose and galactose. Fats in bovine milk include both saturated and unsaturated fatty acids, with the majority being saturated. On average, whole bovine milk contains about 3.5% fat, while reduced-fat milk (2% milk) contains about 2% fat, and fat-free milk (skim milk) contains less than 0.5% fat. The main protein in bovine milk is casein, which makes up about 80% of the total protein content. Bovine milk also contains whey protein, which makes up ˜20% of the protein content. Bovine milk also includes lipoproteins. While high density bovine lipoproteins can be removed through centrifugation, the density of low-density lipoproteins (LDLs) lies between 1.02-1.06 g/ml-close to the density of exosomes, which ranges from 1.04-1.21 g/ml. This relative similarity in particle density poses a challenge to exosome separation for LDLs.

Common exosome isolation techniques include ultracentrifugation (UC), size-exclusion chromatography (SEC), ultrafiltration (UF), precipitation, and immunoaffinity (IA) capture.

Ultracentrifugation (UC) currently remains a widely used technique for the isolation of EVs. The most common exosome isolation method utilizing UC is differential centrifugation, wherein collected samples are centrifuged at various speeds to remove cells, cellular debris, and macromolecular proteins (Yu et al., 2018). Differential centrifugation/ultracentrifugation is effective for the isolation of exosomes from less viscous samples, such as conditioned culture medium, urine, etc. However, the technique is not scalable, time-consuming, labor-intensive, and heavily instrument-dependent (Yu et al., 2018). Moreover, centrifugation methods cannot separate LDL from exosomes, as LDL have similar densities and particle sizes as exosomes.

Size-exclusion chromatography (SEC), also known as gel filtration chromatography, separates exosomes from other macromolecules with different sizes or hydrodynamic diameters. In SEC, samples pass through a chromatographic column packed with beads containing pores of the desired size distribution, and particles are separated by size due to their different elution times. Hybrid resins involve multi-chromatographic separation modes have been developed. Capto Core 700 from Cytiva® is a multimodal chromatography resin designed primarily for the intermediate purification and polishing of viruses and other large biomolecules. It employs a novel core bead technology combined with a multimodal, octylamine ligand. This design imparts the resin with dual functionality: size exclusion and binding properties. The core bead technology allows for efficient capture of contaminants while allowing the target molecules to be collected in the flow-through. While SEC can result in EV fractions with high purity, it increases sample volume post separation. Furthermore, it is difficult to separate exosomes from LDLs, which have similar size profiles.

Exosomes can also be obtained and purified through chemical precipitation agents such as polyethylene glycol (PEG). The precipitate can then be isolated through low-speed centrifugation or filtration. Samples are mixed with the PEG solutions, and the protein in the sample aggregates and precipitates. Exosomes extracted by this method are highly uniform in size, and the method is frequently used for the extraction of exosomes from small samples, such as serum samples, or other samples with high protein concentrations. However, certain contaminants such as lipoproteins may still coexist with the exosomes, thus impairing their subsequent analysis.

Another kind of precipitation is performed by adjusting buffer pH to the isoelectric point of various proteins without adding macromolecules such as PEG and cellulose. At the isoelectric point, protein molecules are neutrally charged and have lower solubility, allowing them to precipitate. The precipitated proteins can then be removed by either filtration or centrifugation. However, milk, and particularly bovine milk, contains large amounts of casein and whey proteins with different isoelectric points. Casein has a relatively consistent isoelectric point at about 4.6, but the isoelectric point of whey proteins-which have smaller molecular sizes—has a larger range, rendering the precipitation process less effective. Inclusion of PEG in exosome is not always designed and it might require additional steps for quality control and purification. Additionally, the particle sizes of aggregated whey proteins may be close to the membrane pore sizes of the tangential flow filtration (TFF) membranes used to enrich exosomes, so membrane fouling may be a challenge. Effective precipitation process involves higher temperature to denature whey and more acidic pH, an additional step that increase cost while potentially damage exosomes. (Hill et al.)

Ultrafiltration (UF), operated in tangential flow filtration (TFF) mode, has been increasingly used to concentrate or enrich EVs from relatively dilute samples. In TFF, a stream of fluids containing EVs flows parallel to the semipermeable membrane surface rather than perpendicular to the membrane surface. Flow along the membrane surface in a tangential direction helps to reduce protein or particle buildup on the membrane surface, reducing fouling. The two most common membrane filters employed in TFF in bioprocessing industries are hollow fiber filters and flat-sheet cassettes.

TFF ultrafiltration (UF) is often performed alongside diafiltration (DF), a process that can change the buffer or solvent in which exosomes are dissolved. The combined procedure is called ultrafiltration-diafiltration (UF/DF). In diafiltration, fresh or new buffer solution is added into the sample while the original solution permeates through the TFF membrane and leaves the sample during the filtration process. The original buffer gradually turns into new buffer, and the unwanted small impurities that can pass through the membrane are carried away. UF/DF is particularly effective in isolating/enriching EVs from large volumes of biological fluid and can remove smaller proteins through diafiltration to exchange buffers. TFF is frequently used with other technologies, such as SEC. While SEC dilutes EVs, TFF can concentrate SEC eluates and concentrate exosomes post SEC purification.

In tangential flow filtration, the feed solution is continuously circulated through the membrane by a pump. The clarified medium is collected on the other side of the membrane as permeate and the feed solution is recirculated until the desired product yield is achieved. TFF is scalable. Particularly, TFF using reusable ceramic membrane has been used in food & beverage, as well as diary industries for commodity product productions.

Hydrophobic interaction chromatography (HIC) is another chromatography technique that aims to separate biomolecules based on their hydrophobicity. HIC exploits the difference in hydrophobicity between EVs and other impurities. During loading, EVs and other proteins can bind to the hydrophobic solid phase of a chromatography column with a high salt aqueous buffer, an environment that favors hydrophobic interactions. As the high-salt buffer mobile phase passes through, the more hydrophobic molecules or particles bind to the hydrophobic stationary phase, while the hydrophilic molecules and the less hydrophobic molecules are eluted from the column first. The bound hydrophobic molecules can then be eluted from the column by decreasing the salt concentration or by adding a competing ligand in the elution buffer. Most commonly, a decreasing salt gradient is utilized to elute samples from the column. The surface of a low-density lipoprotein (LDL) particle is surrounded by a single layer of phospholipids, free cholesterol, and apolipoproteins, and will elute before exosome, while the outer membrane of an exosome is a phospholipid bilayer that is more hydrophobic in nature (Huang et al., 2021) and will elute later.

Isolation of exosomes from milk is particularly challenging due to the presence of high amounts of fat and various proteins, such as casein and whey. Though fat and high-density lipoproteins can be separated through centrifugation, low-density lipoproteins with particle sizes and densities similar to that of exosomes are particularly challenging to remove. Current solid phase HIC has not been reported to be effective in separating LDLs from milk-derived exosomes, as the solid phase conditions do not present enough separation capability. One reason is that the level of impurities is beyond the capability of HIC without pretreatment. For example, it is possible that an overwhelming amount of proteins prevents exosomes from being properly captured. Another potential reason can be that the solid phase does not have the proper differential hydrophobicity to separate exosomes from impurities. The hydrophobicity might stem from the material of the solid phase and possibly the structural geometry of the solid phase.

Presently, there is no established process that combines methods in order to isolate and purify milk exosomes efficiently and at a large scale to achieve high purity. By developing a process to efficiently isolate EVs from large quantities of biological fluid, industries can extract these nanoparticles for large-scale therapeutic treatments.

Additionally, this process offers a new scalable way to separate whey proteins from casein. The purified casein might offer opportunities to simplify cheese making processes, while the isolated whey proteins can be a product as well.

This invention discloses a new process that combines TFF and protein precipitation for the isolation and purification of milk exosomes and potentially other biological fluids to achieve high purity with other using PEG. Additionally, if further combined with hydrophobic chromatography technologies, it can further remove residual lipoproteins to further improve the purity. If using filtration, it is possible to build an end to end closed sterile processing suitable for cGMP applications.

### SUMMARY

In one aspect, the invention discloses a new process for the isolation and purification of milk-derived exosomes. The process comprises a pH-driven precipitation step to separate casein, followed by tangential flow filtration (TFF) to concentrate exosomes while allowing passage of whey proteins. Additionally, hydrophobic interaction chromatography (HIC) is implemented to remove residual lipoproteins and proteins if higher purity is necessary.

In another aspect, the precipitation step involves adjusting the pH to around 4.6 to reach the isoelectric point of casein. This allows aggregation and precipitation of casein while whey proteins remain in solution. The casein precipitate is then removed by centrifugation or filtration.

In another aspect, tangential flow filtration with specific molecular weight cut off (MWCO) membranes between 300 kd-50 nm is utilized. This enriches exosomes in the retentate, while allowing passage of remaining whey proteins into the permeate for removal. Multiple diafiltration buffer exchanges may be conducted to reduce whey protein levels to achieve high yield exosome isolation and purification.

In another aspect, hydrophobic interaction chromatography (HIC) is conducted on the exosome sample using capillary-channeled polymer, or potentially other optimized resin, tailored to leverage hydrophobicity differences between exosomes and lipoproteins. This facilitates separation of residual lipoprotein and protein contaminants.

In another aspect, the combined precipitation, TFF, and HIC process efficiently isolates exosomes while also fractionating milk components into products like whey protein and purified casein. Additional features include scalability for large volumes, process modularity to combine with other methods, reusable filters, and resins to reduce costs, and achievable automation.

In another aspect, terminal sterilization filtration is done before final formulation and storage. The modular process also allows flexibility, such as alternate hybrid chromatography approaches for residual protein removal from exosome product streams.

### BRIEF DESCRIPTION OF THE DRAWINGS

The novel features believed to define the illustrative embodiments are detailed in the appended claims. To fully comprehend these embodiments, along with their preferred usage, objectives, and detailed descriptions, one should refer to the comprehensive description of one or more examples of these embodiments, as provided in this disclosure. This understanding is further enhanced when considered alongside the accompanying drawings, wherein:

FIG. 1 shows a flow diagram outlining the key steps for isolation and purification of exosomes as described in the invention.

FIG. 2 shows particle size distribution analyzed by dynamic light scattering (DLS) for exosome samples isolated using hollow fiber tangential flow filtration membranes of varying molecular weight cut-off (MWCO).

FIG. 3 shows the separation and elution profiles during hydrophobic interaction chromatography using a capillary-channeled polymer resin stationary phase to purify exosomes from residual proteins and lipoproteins after tangential flow filtration.

FIG. 4 illustrates a tubular TFF filter with tube inserts within the flow channel to improve flux and reduce fouling for exosome concentration, purification as well as buffer exchange.

FIG. 5 shows a tangential flow filtration system configuration allowing interim back flush of TFF membrane to reduce membrane fouling as well membrane regeneration and regain fluxes during TFF operation.

FIG. 6 presents nanoparticle tracking analysis (NTA) results for isolated exosomes after 200,000× dilution.

### DETAILED DESCRIPTION OF PREFERRED EMBODIMENTS

The following sections provide a detailed description of the preferred embodiments of the present invention for isolating and purifying milk-derived exosomes. The descriptions and examples are meant to illustrate and enable the invention but should not be interpreted as limiting the scope of the invention.

It should be understood that various modifications and adaptations to the described methods and systems fall within the spirit and scope of the invention. The invention is amenable to alterations and enhancements through routine optimization and experimentation by those skilled in the art based on the disclosure provided herein.

This detailed description first covers preferred steps and processing components involved in the invention. Thereafter, specific implementation examples are described to demonstrate feasibility and effectiveness. Further, potential variations and adaptations within the scope of the inventive concept are noted. Diagrams referenced in the text are provided to visually outline the invention details.

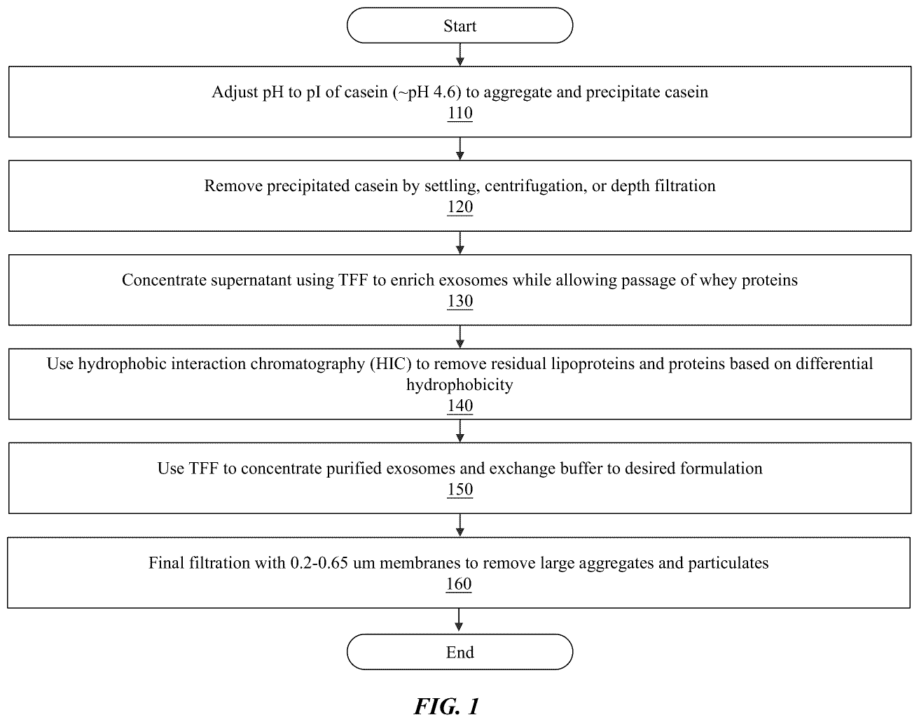

In one aspect, the FIG. 1 provides a flow diagram outlining the preferred essential process steps involved in the integrated milk exosome isolation and purification invention. Beginning with selective aggregation of caseins, the diagram traces the operations of precipitation removal, exosome enrichment coupled to whey protein elimination using tailored tangential flow filtration, hydrophobic chromatography separation of vesicular exosomes from lipoproteins, and final concentration as well as buffer exchange to formulated products. The modular methods enable high exosome purity and recovery, while also allowing fractionation of caseins and whey proteins as by-products. Therefore, FIG. 1 summarizes the critical innovations regarding this integrated precipitation-filtration-chromatography process for obtaining purified milk exosomes at high scale.

Provided as step 110 is the adjusting the pH of skim or fat-free milk to around 4.6, which represents the isoelectric point (pI) of the casein family of milk proteins. An acid such as hydrochloric acid (HCl) is used to lower pH, with gentle mixing for uniform pH change across the milk volume. The casein proteins carry net positive or negative charge depending on whether the pH is lower or higher than their pI value. At pH 4.6, the casein proteins have a neutral net charge and thus aggregate through hydrophobic interactions as well as loss of solubility. These casein aggregates gradually precipitate out from the milk over a timeframe of 15 minutes up to 2 hours. The step 110 may be necessary to selectively separate caseins from other milk components including remaining whey proteins which remain partially soluble at this pH condition.

Further, and as shows as step 120 is the removal of the precipitated casein curd from the residual milk liquid containing disolved proteins like whey along with milk fat globules and exosomes. In preferred aspects, the process employs filtration using a filtration bag with a pore size rating of 2-10 microns to retain the casein flocs while allowing passage of soluble components. Alternatively, centrifugation at appropriate g-force and time can also achieve phase separation, with transparent supernatant collected subsequently. This clarified milk permeate contains whey proteins, some residual caseins, fat globules, exosomes and other milk components. Additional depth filtration is optionally employed if the liquid clarity is insufficient, indicating leftover casein particulates. The casein-rich filter cake can be harvested as a product stream if desired.

On the other hand, the step 130 may utilize tangential flow filtration (TFF) operated in ultrafiltration mode to generate a concentrated residual milk retentate enriched in exosomes, while allowing diafiltration mediated passage of water, lactose, minerals and importantly the remaining whey proteins into the filter permeate. The selectively porous TFF membrane channels have a molecular weight cutoff rating between 300 kilodaltons to 50 nanometers, optimized to transmit dissolved proteins and small molecular weight components specifically into the permeate. A concentration factor of 10-100 can is achieved ultimately by recirculating the residual retentate multiple times across the TFF membrane. Concurrently, diafiltration buffer exchange is also performed by addition of fresh buffer to gradually replace the residual small molecular components with the buffer. A buffer exchange number between 6 to 20 is used, employed either PBS or formulations containing protective osmolytes like trehalose.

In some exemplary aspects, the process step 130 may precede an optional intermediary filtration step using 0.2-0.65 μm pore size membranes, executed after the initial TFF enrichment of exosomes but before subsequent chromatography operations. This is implemented to remove large lipidic globules, particulate impurities, and bacteriological bioburden if any. In one tested iteration, hydrophilic PVDF syringe filters of 0.45 μm pore size were utilized for sterile filtration of isolated exosome batches. This ensured absence of interfering particulates ahead of analytics and assessment.

The sterilized exosome sample can already satisfy purity requirements for certain applications like dermo cosmetic preparations, where trace lipids and proteins may be acceptable. However, higher purity is necessitated for applications related to drug delivery and cell signaling studies. Additional chromatography polishing is hence conducted not just for removal of lipoproteins and proteins through fractionation, but also further sterilization against endotoxins and viruses.

It is preferred that the subsequent step 140 applies hydrophobic interaction chromatography (HIC) to fractionate milk exosomes from residual lipoproteins and proteins in the TFF enriched stream, based on relative hydrophobicity differences amongst vesicular and proteinaceous components. As the precipitation and TFF results in considerable depletion of caseins and elimination of whey proteins, the conditions are more suitable for HIC to leverage binding affinity differences between single lipid layer lipoproteins and the double phospholipid bilayer enveloping exosomes while potentially increasing processing capacity of HIC.

Multiple resin chemistries are evaluated for the HIC phase, including nonpolar polystyrene divinylbenzene substrates like BioBeads SM-2 adsorbents, phenyl functional media, as well as newer capillary channeled polymer fiber columns. The latter most effectively provide differential binding platforms that selectively retain lipoproteins initially followed by more hydrophobic exosomes subsequently.

For capillary channeled phase HIC, the TFF concentrated exosome sample solubilized in 2M ammonium sulfate salt buffered at pH 7.4, is loaded onto a CCP column as illustrated in (Huang et al., 2021) equilibrated with the same high salt medium that induces hydrophobic associations. Step gradient elution with 25% glycerol and ammonium sulfate combination detaches proteins and lipoproteins first in flowthrough and initial wash volumes. This is followed by focused elution of the retained exosomes using a 50% glycerol modified ammonium sulfate mobile phase. The exosome eluate is thus purified away from the lipidic and protein impurities present earlier during the gradient. However, it still requires intermediate processing ahead of final filtration and formulation.

Further in the process is a step 150, which may utilize the principle of tangential flow filtration, however now employed on the purified exosome eluate fraction from the HIC column to firstly concentrate these dilute vesicles by a factor of 5-10×, followed by buffer exchange into an optimized storage medium. Often a PBS pH 7.4 buffer with 25 mM trehalose as an osmo-protectant and preservative is used. A total buffer exchange number ranging between 3 to 10 is sufficient for a complete transition the HIC elution buffer components into the storage formulation. The TFF membrane molecular weight cutoff rating used in this step ranges from 100 kilodaltons to 50 nanometers.

The process step 160 in the integrated process may entail sterile filtration of the formulated exosome product solution through hydrophilically modified 0.45 micrometer pore size membranes, example polyvinylidene fluoride (PVDF) or polyethersulfone (PES) units having low protein binding properties. This filtration barrier ensures removal of any large particulate impurities like residual lipids and protein multimers, bioburden organisms, while enabling unhindered passage of the 50-200 nm sized exosomes into the final sterile formulation product. This sterile grade filtrate constitutes the purified, concentrated, and optimized vesicular formulation for applications like drug delivery and regenerative medicine.

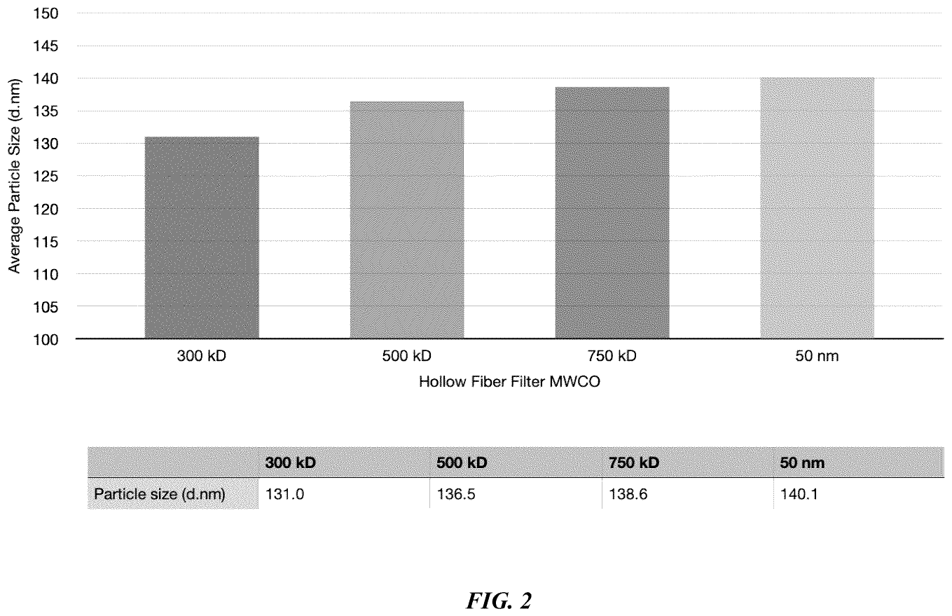

The embodiment of FIG. 2 illustrates particle size distribution profiles for bovine milk exosome samples purified via tangential flow filtration (TFF) operations using hollow fiber ultrafiltration membranes of varying molecular weight cut off (MWCO) ratings. The TFF retention of particles relies on molecular size relativity to pore dimensions rather than absolute particle size cut-offs. By tailoring membrane MWCO from 300 kilodaltons to 50 nanometers, size based selective permeability is achieved, resulting in differential transmission of lipidic, protein and vesicular components. This in turn impacts the hydrodynamic diameter of obtained exosomes following tenfold buffer exchanged concentration.

According to one embodiment, the disclosed process may be implemented for isolation of bovine milk exosomes through the following sequential procedure. Fat-free milk is pH adjusted to 4.6±0.1 using hydrochloric acid addition under gentle agitation, prompting casein aggregation and curd formation over 15 minutes to 2 hours. The casein flocs may be separated by filtration through a 2-10 μm pore size scalable bag, allowing passage of soluble whey proteins along with lipidic globules and exosomes. The transparent casein-depleted permeate may be loaded into a TFF recirculation reservoir and concentrated utilizing 300 kd-50 nm MWCO Hollow fiber ultrafilters under shear of 3000-6000 s⁻¹ and 2-6 psi TMP. Concurrent extensive 10× PBS buffer diafiltration may remove permeating whey proteins while obtaining 10-15× enrichment of retentate exosomes. The TFF purified exosome sample after 0.45 μm sterile filtration may then undergo HIC on capillary channeled polymer (CCP) columns for separation from trace lipoproteins. Column equilibration and sample loading may use 2M ammonium sulfate buffer to leverage hydrophobic interactions. Step elution involving glycerol displaces proteins and lipoproteins before subsequent high purity exosome elution.

A final TFF concentration and buffer exchange prepares formulated dosing aliquots. This small scale embodiment thus reflects the major precipitation, filtration and chromatography innovations for harnessing high milk exosome yields with by-product protein fractionation.

The tangential flow filtration outcomes from the described embodiments related to exosome recovery and morphology are summarized in Table 1 & FIG. 2.

Dynamic light scattering (DLS) using Malvern Zeta Sizer Nano Series analysis was done on 0.2 micron sterile filtered aliquots of TFF purified exosome fractions originating from 300, 500, 750 kDa and 50 nm MWCO ultrafilters. Number weighting converts the intensity weighted size distribution that accounts for greater light scattering from larger particulates. As the membrane pore size increases from 300 kDa to 50 nm, an increase in mean isolated exosome diameter is noted ranging from 131.0 nm to 140.1 nm. In contrast, the less restrictive 50 nm ceramic membrane transmits smaller vesicles and resulting isolated exosomes with larger sizes.

By tailoring the MWCO from 300 kilodaltons to 50 nanometers, the mean isolated vesicle diameter can be tuned as analyzed by dynamic light scattering (DLS) using a Malvern Zeta Sizer Nano Series instrument. This results presented a way to fine tune the particle sizes of exosomes for different applications. Therefore, the Table 1 shows the impact of hollow fiber MWCO to the particle sizes of isolated exosome.

TABLE 1
Tangential Flow Filtration Outcomes

| | 300 kD | 500 kD | 750 kD | 50 nm |
|---|---|---|---|---|
| Particle size (d · nm) | 131.0 | 136.5 | 138.6 | 140.1 |

The determined concentration of exosomes is normalized to the concentration in the starting volume of milk, and results is shown in Table 2. It's noted as the final sample volume used in the studies is less than 2 ml, and loss of exosome sample in filtration and sample transfer can be substantial and the solution about EV yield vs hollow fiber filter MWCO should not be made from this data.

TABLE 2
Normalized EV Yield vs. Hollow Fiber MWCO

| | |
|---|---|
| Hollow fiber filter | Normalized EV yield |
| MWCO | (particles/mL) |

| | | |
|---|---|---|
| 300 | kD | 5.60E+11 |
| 500 | kD | 7.60E+11 |
| 750 | kD | 9.20E+11 |
| 50 | nm | 4.50E+11 |

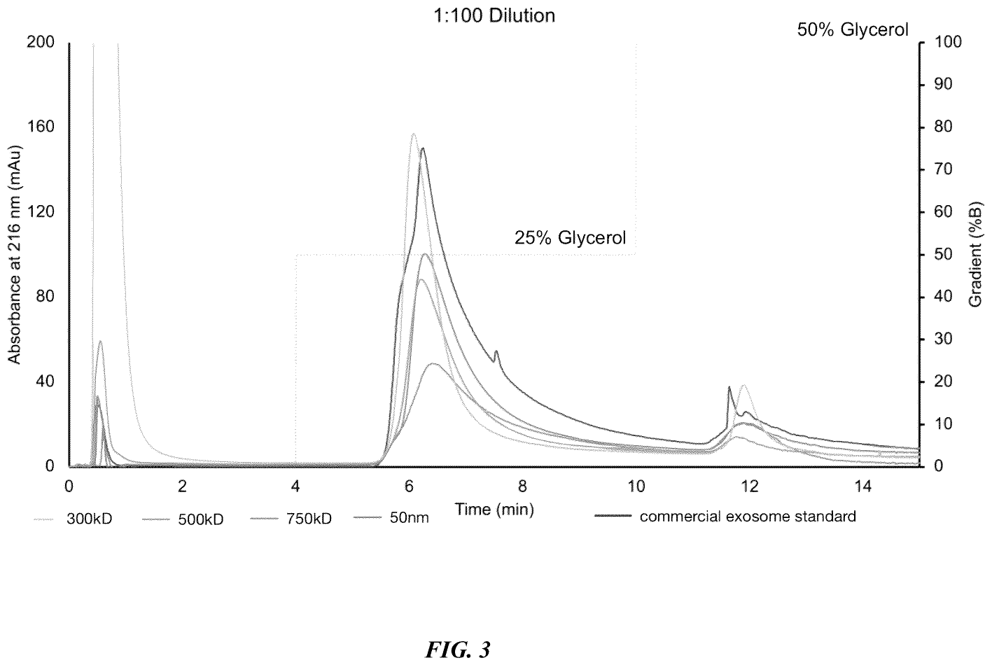

The purification efficacy of isolated exosomes in the embodiment is further analyzed by hydrophobic interaction chromatography which leverages differential surface hydrophobicity. The TFF enriched vesicles solubilized 1:1 in 2M ammonium sulfate of 20 um is loaded onto an PET capillary channeled polymer columns following previously established procedures (Huang et al.). Initial column flowthrough and intermediate wash fractions correspond to unbound hydrophilic protein impurities. Subsequent elution steps lead to mild hydrophobicity lipoproteins and lipidic globules detaching initially. The stronger interacting vesicular exosomes finally elute under high chaotrope concentrations, detected at 216 nm absorbance. The FIG. 3 shows the separation and elution profiles during hydrophobic interaction chromatography using a capillary-channeled polymer resin stationary phase to purify exosomes from residual proteins and lipoproteins after tangential flow filtration.

The figure summarizes the hydrophobic interaction chromatography profiles between the embodiment isolated exosome sample and an affinity-purified commercial exosome standard. The negligible initial non-vesicular protein and lipoprotein elution followed by a prominent spheroidal vesicle fraction peak in the embodiment sample matches the control chromatogram. This demonstrates comparable purity by the tailored precipitation-TFF invention combination to established affinity capture methods.

For the 750 kDa HF tract exosomes, a 100 kilodalton MWCO centrifugal ultrafiltration concentrates 10-fold and exchanges into PBS/25 mM trehalose storage buffer, attaining roughly 70% yield for the HIC steps. Final 0.45 μm sterile filtration ensures absence of particulates. While this reflects one embodiment tailored for small batches, the underpinning HIC phase can linearly scale to large volumes in conjunction with appropriately dimensioned CCP columns or alternate resins once available.

Additionally, the entire tailored precipitation-TFF invention process is adaptable to large scale cGMP bioprocessing leveraging robust and regenerable ceramic ultrafiltration membranes already prevalent in several bioproduction industries for their economy. Though this invention employs small CCP columns, the CCP chromatography step is also scalable once larger column is available. The integrated invention thus enables cost-effective, reproducible and highly pure isolation of milk exosomes at manufacturing scale levels.

### Cases of Implementation at Larger Scale for Isolation of Exosome

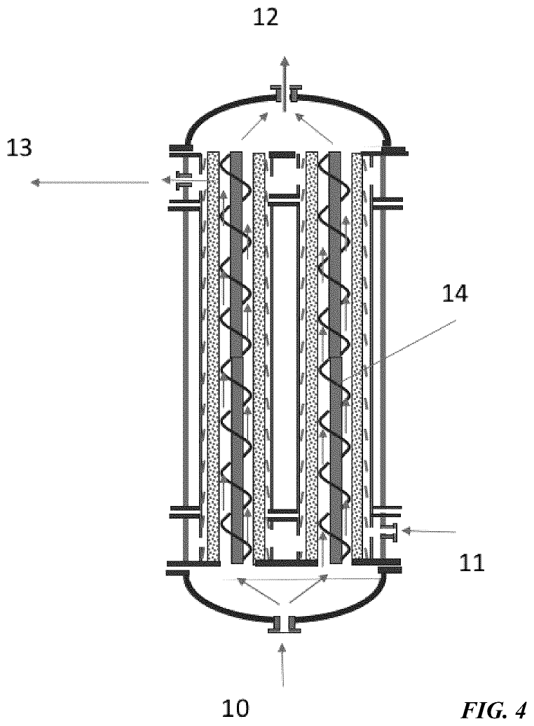

The FIG. 4 illustrates a tangential flow filtration (TFF) device that enables back pulsing for enhanced efficiency and process capacity at larger processing. The feed 10 enters the tubular ceramic membrane channel containing spiral ridge inserts 14. These inserts induce turbulence minimizing fouling. The retentate 13 flows axially facilitating concentration of retained exosomes while permeating whey proteins 12 exit through the filtrate port. The integrated TFF system thereby allows sustained diafiltration concentration of milk exosomes through the membrane channel while mitigating flux loss through back pulsing. The rigid porous ceramic construct with bio-inspired ridge inserts (14) enables consistent high throughputs competing with single-pass cassettes. In TFF processes, when the fouling occurs, the disclosed TFF system allows for back flushing and cleaning of membrane in line. The back flushing can use PBS buffer or other solution cleared of particles and with proper composition compatible with the sample. Additionally, membrane cleaning reagents such as Alconox® membrane solution can be delivered for back flushing and membrane cleaning followed by buffer rinsing. By preventing polarization and managing fouling through back wash cycles, the device facilitates large scale milk batch exosome isolation.

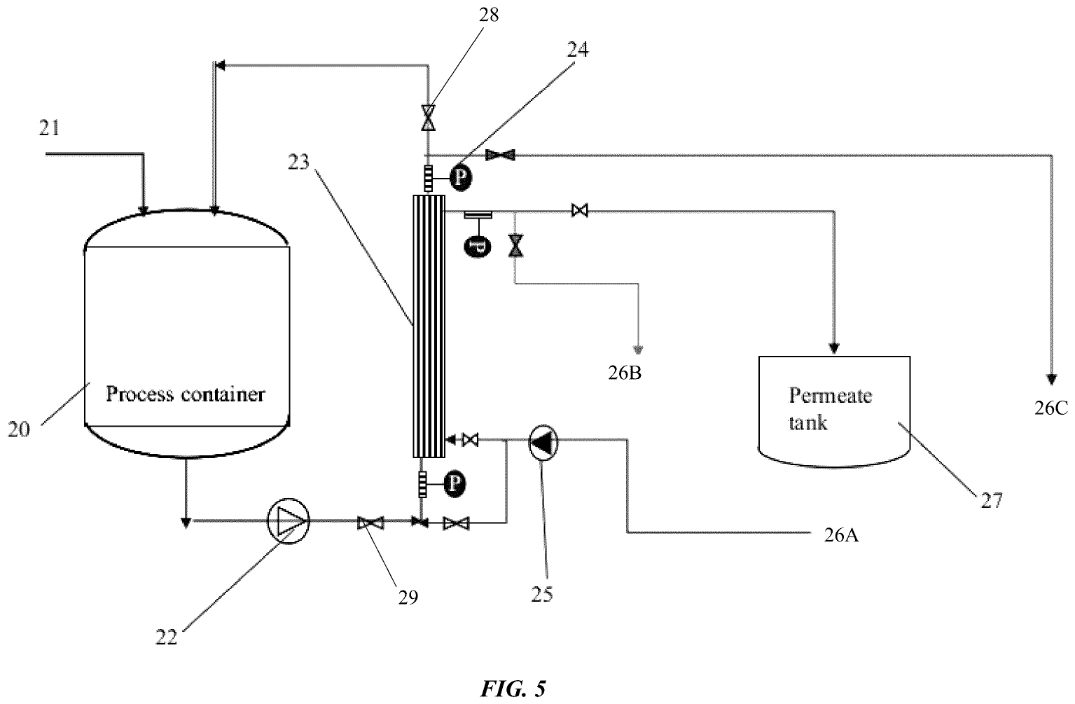

On the other hand, the FIG. 5 shows a tangential flow filtration system configuration allowing filter regeneration and back flushing for maintaining closed system sterility during process steps. Specifically, the figure illustrates a tangential flow filtration (TFF) system with integrated provisions for membrane regeneration through back flushing and regeneration during milk exosome isolation steps. The core recirculation reservoir 20 contains the process fluid for concentration/diafiltration by pumping using pump 22 through the TFF filter module 23, which may be passed through a feed valve 29. Pressure sensor 24 allows monitoring transmembrane pressure. Permeate collects in a storage tank 27 during concentration. A flushing pump 25 permits independent cleaning, flushing, or sterilizing fluid circulation like 0.2 μm filtered sodium hydroxide, Alconox® cleaning solutions, PBS buffer. The back flushing and cleaning solution exits 26A, 26B and 26C are also shown. A permeate valve 28 and feed valve 29 may be provided as illustrated to isolate the TFF device from the exsome solution during back flushing, membrane cleaning and flushing/rinsing.

Additionally, the configuration allows connections for feeding supplementary feeds 21 like buffer, process additional required additives into the recirculation loop without exposure to atmosphere. The flushing connections joining at manifold points near the reservoirs allow basic buffer sanitization of the contact surfaces. The flushing solution can exit the flush path.

It is noted if the liquid entering in the flow path were sterile filtered, including back flushing buffer, cleaning fluid, or additional flushing buffers, then the capability for closed sterile addition of supplementary feeds 21 alongside independent fluid flushing prevents microbial ingress while enabling pH adjustment, buffer conditioning or additive dosing. By ensuring aseptic interconnectivity, the innovation enables implementations under cGMP biomanufacturing environments for milk exosome fractionation.

In one embodiment, the TFF system used is a Spectrum Labs KR2i (Spectrum SYR2-U20) counter top automated TFF system which includes a main recirculation pump, and up to two auxiliary pumps for buffer addition or delivering flushing buffers from one of permeate port. The flushing buffer can exit for another permeate ports or through retentate side. If a not-compatible buffer such as Alconox® cleaning solution is used for back flushing, the flushing should not be allowed in contract with exosome samples and enter into the process container during the processes. The TFF process should be suspended while the membrane is been generated and before flushed with proper compatible buffer such as PBS buffers.

Additionally the unit operations enable not just diafiltration based concentration to obtain exosome enrichment, but also allows intermittent back pulsing and membrane regeneration. In the latter, recirculation pump 22 is stopped and valves at the feed line 29 and retentate 28 are also closed to isolate the filters from the process samples. The top permeate side ports are temporarily closed and the flushing buffer 26A is instead pumped in through the bottle permeate ports into the filter and flow in reverse direction through the membrane back to retentate side exiting as 16C, which a part of flushing buffer can exit as 26B for the top permeate port. The high shear reverse filtration serves to hydraulically wash the membrane, dislodging any forming fouling layer while still enabling sustained concentration. In some cases, the back pulsing last for about 1 minutes with pressure about 10 psi.

### 3 Gallon Batch Demonstration

In one aspect, disclosed process is scalable to a larger volume as shown by a 3 gallon fat-free milk batch experiment. Hydrochloric acid addition (1 N) lowers pH to 4.6 prompting casein precipitation. A 10 liter capacity 5 μm rating PE filter bag (Felt Liquid Filter Socks Bag from Amazon Honritone brand) then separates the casein flocs, with recirculation employed until a clarified filtrate for protein solution with suspended exosomes, lipids and whey proteins is obtained. The casein filter cake offers a potential byproduct stream.

After the bag filtration, a 207 cm2 double layer 0.2-0.2 μm pore depth filter (Cobetter part number: Part No. L05SSCSDD02PC) polishes the pre-clarified stream ahead of ceramic UF/DF concentration, reducing particulate interference that can foul membranes. About 2 L of PBS buffer is used to pre-rinse and condition the depth filter before sample loading.

The previous steps resulted 8.5 L of whey protein solutions, which was concentrated and purified using a Tanda Biotech ceramic membrane TFF filter (L30-05u-30-04-02-03-PP). The filter uses a 19-channel ceramic membrane tube with channel ID of 4 mm each. An insert is included in each flow channel. The length of the membrane tube is about 30 cm, and working surface area is about 600 summing to ˜600 cm2 area. The key novelty is tube inserts made of 2.5-2.7 mm diameter polymer rod (such as nylon or polypropylene) with spiral fins with OD about 3.5 mm. The inserts can reduce the cross section of liquid flow channel and can promote velocity transitions that improve surface shear and mass transfer. The 3.5 OD insert fins hence regulate axial flow as well as introduce controlled turbulence minimizing polarization and fouling.

The internal channel inserts thus facilitate increased shear protecting exosomes from shear damage while gaining anti-fouling capability allowing multistage concentration. The high area ceramic construct thereby serves as a scalable bioprocessing module competing with single use cassettes in performance while offering reusability benefit. This integrated fouling reduction and pulsatile back flush innovation enables uncompromised isolation of milk exosomes at manufacturing scales.

By using a Spectrum Labs KR2i system (SYR2-U20) with size 17 Pharmapure tubing and operated at an average recirculation rate of 1.2 liter per minute (LPM) and 2-2.5 psi, the cleared supernatant was concentrated to bout 140 ml, about the holdup volume of the retentate flow buffer. PBS buffer can be used in buffer exchange to wash away remaining whey proteins following the concentration. In this testing, 9 times of standard PBS buffer exchanged was first conducted after 60 times of concentrations from about 8.5 L to 140 ml in the stage concentration.

If higher concentrations are desired, the exosome solution can be further concentrated in a second stage. As an example, 140 ml was additionally concentrated to 70 ml using a Tanda Biotech 66 m2 surface area ceramic membrane (L10-05U-12-08-05-07-PP) in one trial.

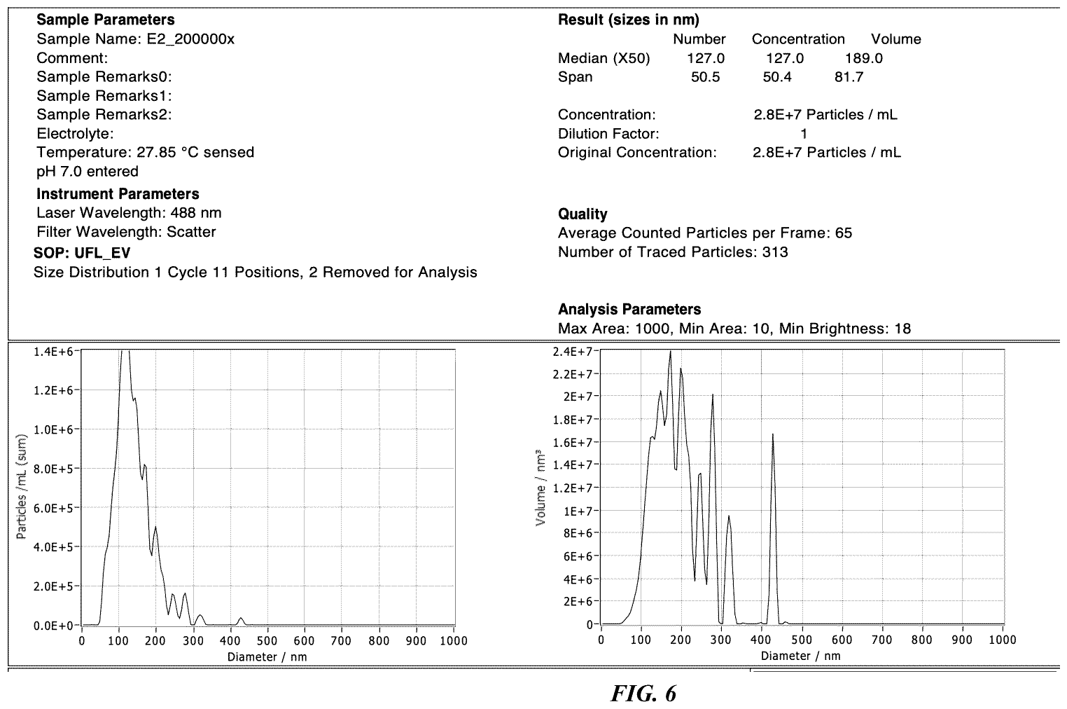

The NTA determined particle concentration after 200,000× dilution is 2.8×10⁷ particles/ml as shown in FIG. 6. The original isolated exosome sample concentration is 5.6×10¹³ particles/ml in the undiluted preparation. Accounting for dilution and concentration, this is equivalent to ˜7×10¹¹ particles/ml in the original milk. The reduced size from NTA is probably resulted from extra clarification steps using depth filter with size rating of 0.2-0.4 um.

The above mentioned 3 gallon process is thus uniquely geared not just for isolate purification, but also seamless scale-up leveraging common bioprocessing unit operations. Key innovations in tailored precipitation and membrane selectivity supplementation provide inherent scalability lacking in extant isolation techniques. Having validated primary casein removal, concentration of exosome and buffer exchange to remove whey protein steps remain analogous with inbuilt scalability for the large starting milk batches. With appropriately expanded modules, cGMP grade exosome generation from hundreds to thousands of liter batches is feasible.

As the current invention leverages the scalability and large processing capabilities of ceramic membrane TFF systems along with existing dairy industry infrastructure; we foresee the isolation can be scaled to produce hundreds of thousands of liters sized batches—providing sufficient bovine exosomes for commercial scale production.

In some embodiments, mixed mode chromatographic resins can be used rather than just hydrophobic interaction media. One option is GE Life Science's Capto Core 750 designed for separating larger exosomes particles from smaller protein contaminants.

Certain embodiments incorporate a protocol recommended by Cytiva, which involves loading the clarified exosome sample onto the Capto Core 750 column at the recommended flow rate.

In certain aspects, a column may be eluted with PBS buffer as the equilibration buffer to collect exosomes, which cannot penetrate into the core due to their larger size. Since the Capto Core 750 resin is designed to allow larger particles like exosomes to flow through while retaining smaller proteins, the exosomes elute in the initial column flow-through and wash volumes.

In certain aspects, the exosome-containing fractions may be concentrated using ultrafiltration or other suitable techniques and buffer exchanged as required. In some cases, 3× buffer exchange into PBS with 25 mM trehalose may be performed on the isolated exosomes ahead of lyophilization when no further purification was needed.

In certain aspects, the implementation in the disclosure illustrates a typical chromatography-based process for enhancing exosome purification after initial isolation.

Certain embodiments incorporate hybrid resin to remove residue proteins using solid phase extraction mode, by mixing the exosome samples with resin such as Capto Core 750, and then remove the resin particle by filtration or simply by aspirating the supernatant after the resin particle are settled.

Certain embodiments incorporate one can spike in higher concentration of Trehalose solution in the final exosome products instead of using buffer exchange.

This application represents model implementations of the method, while variations in HIC chromatography solid phase and liquid phase conditions to remove residual protein impurities including lipoproteins are considered within the scope of this invention. Additionally, alternate TFF membrane materials like flat sheet cassettes or hollow fiber modules also fall within the approach scope.

This invention utilizes bovine milk to demonstrate the methods for combined exosome isolation and fractionation of major milk components such as whey proteins, caseins and exosomes. It may be possible to additionally isolate other minor components in these fractions, including lactoferrin, albumin etc.

The disclosed method(s) demonstrates selective casein precipitation at its isoelectric point without requiring pH adjustment to neutral before TFF. This is adaptable for isolating exosomes and separating major milk components from other dairy sources as well. It would involve adjusting the pH to the pI of the respective target protein to induce controlled precipitation, followed by separation using filtration/centrifugation/gravity settling ahead of TFF. To fractionate different components, multiple precipitation-clarification-TFF cycles may need to be implemented in a tailored sequential process.

What is claimed is:

1. A method for the separation of multiple proteins and exosomes from biological fluids comprising:
removing fat from the biological fluid via centrifugation;
adjusting milk pH to the pI of a target protein to aggregate and precipitate said protein;
using filtration or centrifugation to separate precipitated protein from supernatant;
using tangential flow filtration (TFF) uses 300 kD-50 nm MWCO membrane to concentrate exosomes, further purify and exchange buffer; and
optionally doing final filtration with 0.2-0.65 μm sterile filters.

2. The method of claim 1, wherein the biological fluid is bovine milk, the target proteins are casein and whey proteins, further comprising:
precipitating casein at pH 4.0-5.0;
removing casein via filtration or centrifugation, with optional secondary clarification;
using TFF device to concentrate exosomes and remove residual whey proteins via buffer exchange; and
optionally doing final 0.2-0.65 μm filtration.

3. The method of claim 1, further comprising chromatographically removing residual proteins from isolated exosomes.

4. The method of claim 2, further comprising using hybrid chromatography resin leveraging size exclusion and protein adsorption to reduce remaining proteins.

5. The method of claim 1, wherein TFF uses 300 kD-50 nm MWCO ceramic membranes, optionally with tube inserts.

6. The method of claim 2, wherein TFF uses 300 kD-50 nm MWCO ceramic membranes, optionally with tube inserts.

7. The method of claim 3, using hydrophobic interaction chromatography with capillary-channeled polymer stationary phase.

8. The method of claim 1 using 300-50 nm MWCO tubular TFF membranes and intermittently back pulsing at ≥3× average flux to control fouling and enhance efficiency.

9. The method of claim 5 or 6, using multiple TFF stages including single pass operations.

10. The method of claim 1, further comprising final TFF concentration and buffer exchange of purified exosomes into optimized storage buffer.

11. A system for isolating and purifying milk-derived exosomes comprising:
a precipitation vessel configured to separate casein at milk pH of 4.0 to 5.0;
a filtration device or centrifuge to remove precipitated casein;
a tangential flow filtration unit with 300 kd to 100 nm molecular weight cut off membrane; and
a hydrophobic interaction chromatography column optimized for separating lipoproteins from exosomes.

12. The system of claim 11 further comprising one or more sterile filters with 0.2 um to 0.65 um pore size for terminal filtration.

13. The system of claim 11 wherein the tangential flow filtration unit comprises channel inserts or spacer elements to improve flux and hydraulic efficiency.

14. The system of claim 11 configured for recycling filtered casein and whey protein fractions as by-product streams.

15. The system of claim 11 further comprising a 0.2 um to 0.65 um sterile filter for terminal filtration of the purified exosomes.
